(19) United States
(12) Patent Application Publication
(10) Pub. No.: US 2026/0341459 A1
(43) Pub. Date: Oct. 29, 2026

(54) METHODS AND SYSTEMS FOR PREDICTING THE PROBABILITY AND DURATION OF ICU PATIENT VENTILATION

(71) Applicant: KONINKLIJKE PHILIPS N.V., EINDHOVEN (NL)

(72) Inventors: LOUIS NICOLAS ATALLAH, BOSTON, MA (US); LUDMILA BROCHINI, EINDHOVEN (NL); PAMELA JAYNE AMELUNG, CLARKSVILLE, MD (US); OMAR BADAWI, LUTHERVILLE TIMONIUM, MD (US); XINGGANG LIU, BALTIMORE, MD (US)

(21) Appl. No.: 19/110,198

(22) PCT Filed: Sep. 5, 2023
(86) PCT No.: PCT/EP2023/074219

§ 371 Date: Mar. 10, 2025

Related U.S. Application Data
(60) Provisional application No. 63/406,453, filed on Sep. 14, 2022.

(51) Int. Cl. G16H 20/40; G16H 40/20; G16H 50/70

(52) U.S. Cl. CPC G16H 20/40; G16H 40/20; G16H 50/70

(57) ABSTRACT

The present disclosure relates to methods and systems for predicting intensive care unit (ICU) ventilation. In certain embodiments, the methods described herein include: providing an ICU ventilation prediction system; obtaining a plurality of records for a patient in an ICU covering at least a first time period; extracting a plurality of different defined ICU prediction features for the patient; analyzing the extracted plurality of different defined ICU prediction features using a trained ICU ventilation prediction model; generating a likelihood of ICU ventilation for the patient, wherein the likelihood of ICU ventilation comprises both (i) a prediction of invasive and/or non-invasive ventilation of the patient in the ICU, and (ii) a predicted duration of invasive and/or non-invasive ventilation of the patient in the ICU; and presenting the generated likelihood of ICU ventilation for the patient via a user interface.

### FIELD OF THE DISCLOSURE

The present disclosure relates generally to methods and systems for predicting the need for ventilation support for an intensive care unit (ICU) patient, and more specifically to methods and systems for predicting intensive care unit ventilation using a machine learning model.

### BACKGROUND

Widespread adoption of electronic health records has enabled automated data capturing and propelled predictive risk modeling in a variety of respects and across many different cohorts. In certain settings, risk adjusted predictive modeling has become an essential pillar for measuring outcomes, effective care unit planning, and other benchmarking.

For example, ventilation is an important part of critical patient care, which should be provided at the earliest point of patient need. While mechanical ventilation has proven to be a lifesaving intervention for patients in respiratory distress, delays in intubation can carry significant risk. On the other hand, mechanical ventilation support carries certain risk, such as permanent lung injuries and infection, which can lead to ventilator-associated complications and even death. Additionally, patients requiring prolonged mechanical ventilation have a substantially longer length of stay in the intensive care unit (ICU) and account for a large portion of the cost of care for ventilated patients.

The importance of understanding ventilation needs of a healthcare ecosystem was further highlighted during the COVID-19 pandemic, as hospitals needed to optimize ventilator use for their most vulnerable patients. However, existing methods and systems of predicting a patient's need for ventilator support fail to discriminate between invasive and non-invasive ventilation and do not offer a probability of ventilation for generalized ICU patients, among other shortcomings.

### SUMMARY OF THE DISCLOSURE

Accordingly, there is a continued need for clinical systems that more accurately predict the likelihood that a patient will require ventilation while in the ICU, including whether the patient is likely to require invasive or non-invasive ventilation.

According to an embodiment of the present disclosure, a method for predicting a likelihood of ventilation of a patient in an intensive care unit (ICU) using an ICU ventilation prediction system is provided. The method comprises: providing an ICU ventilation prediction system; obtaining, from an electronic medical records database, a plurality of records for a patient in an ICU covering at least a first time period; extracting, from the obtained plurality of records, a plurality of different defined ICU ventilation prediction features for the patient; analyzing the extracted plurality of different defined ICU ventilation prediction features using a trained ICU ventilation prediction model; generating, from the analysis, a likelihood of ICU ventilation for the patient, wherein the likelihood of ICU ventilation comprises both: (i) a prediction of invasive and/or non-invasive ventilation of the patient in the ICU; and (ii) a predicted duration of invasive and/or non-invasive ventilation of the patient in the ICU; and presenting, via a user interface of the ICU ventilation prediction system, the generated likelihood of ICU ventilation for the patient. The ICU ventilation prediction model may be trained by: (i) obtaining, from an electronic medical records database, a plurality of records for each of a plurality of patients in an ICU covering at least a first time period, wherein at least some of the plurality of patients in the ICU did not receive any ventilation, and wherein at least some of the plurality of patients in the ICU received non-invasive ventilation for a known non-invasive ventilation period, and wherein at least some of the plurality of patients in the ICU received invasive ventilation for a known invasive ventilation period, and wherein at least some of the plurality of patients in the ICU received both invasive and non-invasive ventilation each for a known ventilation period; (ii) extracting, from the obtained plurality of records, a plurality of different health features for each of the plurality of patients; (iii) manually curating, based on the results of extracting, the extracted plurality of different health features to identify the plurality of different defined ICU ventilation prediction features; (iv) training the ICU ventilation prediction model using the plurality of different defined ICU ventilation prediction features for at least some of the plurality of patients; and (v) storing the trained ICU ventilation prediction model.

In an aspect, the prediction of invasive and/or non-invasive ventilation of the patient in the ICU comprises a prediction of one or more of: (i) no ventilation; (ii) invasive ventilation only; (iii) non-invasive ventilation only; and (iv) both invasive and non-invasive ventilation.

In an aspect, the prediction of invasive and/or non-invasive ventilation of the patient in the ICU comprises a prediction of one or more of: (i) ventilation versus no ventilation; (ii) invasive ventilation versus no invasive ventilation; and (iii) non-invasive ventilation versus no non-invasive ventilation.

In an aspect, the extracted plurality of different defined ICU ventilation prediction features for the patient comprises some or all of the features in TABLE 1.

In an aspect, the ICU ventilation prediction system is, or is a component of, a patient data management systems (PDMS) or a patient monitoring system.

In an aspect, the patient is a historical patient.

In an aspect, the ICU ventilation prediction model is a gradient-boosted decision tree model.

In an aspect, the trained ICU ventilation prediction model can analyze the extracted plurality of different defined ICU ventilation prediction features and generate a likelihood of ICU ventilation for the patient when some of the plurality of different defined ICU ventilation prediction features are missing from the obtained plurality of records.

In an aspect, at least some of the extracted plurality of different health features are binned prior to training the ICU ventilation prediction model, and wherein said bins comprise a bin comprising missing data.

In an aspect, the binned extracted plurality of different health features comprise one or more of blood albumin, blood lactate, arterial blood gas pH, arterial blood gas PaCO₂, arterial blood gas PaO₂, PF ratio, and Glasgow Coma Scale (GCS) score.

According to another embodiment of the present disclosure, an intensive care unit (ICU) ventilation prediction system configured to predict a likelihood of ventilation of a patient in an ICU is provided. The ICU ventilation prediction system comprises: an electronic medical records database comprising a plurality of records for a plurality of patients; a trained ICU ventilation prediction model configured to analyze a plurality of different defined ICU ventilation prediction features to generate a likelihood of ICU ventilation for a patient; a processor; and a user interface configured to provide the generated likelihood of ICU ventilation for the patient. The ICU ventilation prediction model can be trained by: (i) obtaining, from an electronic medical records database, a plurality of records for each of a plurality of patients in an ICU covering at least a first time period, wherein at least some of the plurality of patients in the ICU did not receive any ventilation, and wherein at least some of the plurality of patients in the ICU received non-invasive ventilation for a known non-invasive ventilation period, and wherein at least some of the plurality of patients in the ICU received invasive ventilation for a known invasive ventilation period, and wherein at least some of the plurality of patients in the ICU received both invasive and non-invasive ventilation each for a known ventilation period; (ii) extracting, from the obtained plurality of records, a plurality of different health features for each of the plurality of patients; (iii) manually curating, based on the results of extracting, the extracted plurality of different health features to identify the plurality of different defined ICU ventilation prediction features; (iv) training the ICU ventilation prediction model using the plurality of different defined ICU ventilation prediction features for at least some of the plurality of patients; and (v) storing the trained ICU ventilation prediction model. The processor can be configured to: (i) obtain, from the electronic medical records database, a plurality of records for a patient in an ICU covering at least a first time period; (ii) extract, from the obtained plurality of records, a plurality of different defined ICU ventilation prediction features for the patient; (iii) analyze the extracted plurality of different defined ICU ventilation prediction features using the trained ICU ventilation prediction model; and (iv) generate, from the analysis, a likelihood of ICU ventilation for the patient, wherein the likelihood of ICU ventilation comprises both: (i) a prediction of invasive and/or non-invasive ventilation of the patient in the ICU; and (ii) a predicted duration of invasive and/or non-invasive ventilation of the patient in the ICU.

In an aspect, the prediction of invasive and/or non-invasive ventilation of the patient in the ICU comprises: a prediction of one or more of: (i) no ventilation; (ii) invasive ventilation only; (iii) non-invasive ventilation only; and (iv) both invasive and non-invasive ventilation; or a prediction of one or more of: (i) ventilation versus no ventilation; (ii) invasive ventilation versus no invasive ventilation; and (iii) non-invasive ventilation versus no non-invasive ventilation.

In an aspect, the extracted plurality of different defined ICU ventilation prediction features for the patient comprises some or all of the features in TABLE 1.

In an aspect, the ICU ventilation prediction system is, or is a component of, a patient data management systems (PDMS) or a patient monitoring system.

In an aspect, at least some of the extracted plurality of different health features are binned prior to training the ICU ventilation prediction model, and wherein said bins comprise a bin comprising missing data.

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for predicting a likelihood of ventilation, illustrated according to aspects of the present disclosure.

FIG. 2 is a schematic diagram of an ICU ventilation prediction system, illustrated according to aspects of the present disclosure.

FIG. 3 is a flowchart of a method for training an ICU ventilation prediction model, illustrated according to aspects of the present disclosure.

FIG. 4 is a graph showing the relative performance of an inventive ICU ventilation prediction model and two comparative models, illustrated according to aspects of the present disclosure.

FIG. 5 is a graph showing the performance of an inventive ICU ventilation prediction model, illustrated according to aspects of the present disclosure.

### DETAILED DESCRIPTION OF EMBODIMENTS

The present disclosure is directed to methods and systems for predicting intensive care unit ventilation based on clinical features from the first 24-hour stay of a patient using risk models that mitigate different biases. As described herein, the methods and systems reduce the documentation burden to obtain ventilation predictions, and meet and/or exceed accuracy and performance benchmarks for existing models.

The embodiments and implementations disclosed or otherwise envisioned herein can be utilized with any patient care system, including but not limited to clinical decision support tools, among other systems. For example, one application of the embodiments and implementations herein is to improve analysis systems such as, e.g., the Philips® eCareManager Enterprise telehealth products, Philips® Tasy EMR solutions, and Philips® Patient Flow Capacity Suite products, among many others. However, the disclosure is not limited to these devices or systems, and thus disclosure and embodiments disclosed herein can encompass any device or system capable of generated and reporting information about ventilation of a patient.

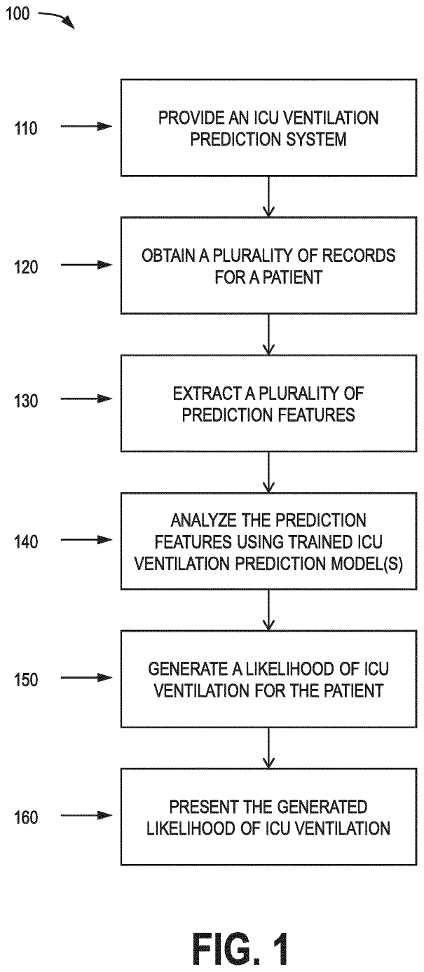

Turning to FIG. 1, a flowchart of a method 100 for predicting a likelihood of ventilation for a patient in an intensive care unit (ICU) using an ICU ventilation prediction system is illustrated according to aspects of the present disclosure. The ICU ventilation prediction system can be any of the systems described or otherwise envisioned herein.

At a step 110 of the method 100, according to an embodiment, an ICU ventilation prediction system 200 is provided. As discussed in greater detail below, the ICU ventilation prediction system 200 can be configured to predict a likelihood of ventilation for a patient. In embodiments, the ICU ventilation prediction system 200 can include a trained ICU ventilation prediction model 264 configured to analyze a plurality of different defined ICU prediction features to generate a likelihood of ventilation for the patient. In some embodiments, one or more ICU ventilation prediction model(s) 264 may be developed using a generalized additive model (GAM) framework, which allows the ICU ventilation prediction model(s) 264 to use non-linear functions of continuous features while maintaining the additivity of multivariate linear regression.

At a step 120, the method 100 includes obtaining a plurality of records for a patient in an ICU. In some embodiments, the plurality of records for the patient may be obtained by the ICU ventilation prediction system 200. In further embodiments, the plurality of records for the patient may be obtained from an electronic medical records database 270A, 270B. For example, the electronic medical records database 270A, 270B may include patient unit stays admitted to ICUs where physiologic, diagnosis, and treatment information (collectively, “medical information” or “medical data”) are captured, charted, or otherwise recorded. That is, the electronic medical records database 270A, 270B can comprise a plurality of healthcare-related records for a plurality of patients, including historical patients and/or patients of current ICU stays.

In still further embodiments, the plurality of records obtained in step 120 can include medical records that cover a first period of time. For example, the plurality of records obtained in step 120 can include medical records that cover the first 24 hours of the patient's stay in an ICU (i.e., the medical data available through the first day of ICU admission), although longer and shorter periods of time are possible.

Alternatively, if medical records for the patient within the first day of ICU admission are not available, the medical records may include, for example and without limitation, medical records covering up to six hours before ICU admission, although longer and shorter time periods are possible.

As such, in various examples, the first period of time can include, for example, the first 24 hours of the patient's ICU stay, only the first 24 hours of the patient's ICU stay, less than 24 hours of the patient's ICU stay, an amount of time (e.g., 1 hour, 3 hours, 6 hours, 12 hours, etc.) preceding the patient's admission to the ICU, and/or some combination thereof.

At a step 130, the method 100 includes extracting a plurality of different defined ICU prediction features for the patient from the plurality of records obtained in step 120. In embodiments, the ICU ventilation prediction system 200 may extract the plurality of different defined ICU prediction features for the patient based on the plurality of records obtained in step 120.

As used herein, the term “defined ICU prediction features” refers to continuous physiologic, diagnosis, and/or treatment information that are defined prior to analyzing the plurality of medical records of the patient using a trained model. In embodiments, the plurality of different defined ICU prediction features can include of the defined prediction features shown in Table 1 below:

TABLE 1
LIST OF ICU PREDICTION FEATURES.

| Feature | Summary Measure | Categorized |
|---|---|---|
| Admission BMI | NA | NA |
| Gender | NA | NA |
| Hours in hospital prior | NA | NA |
| to ICU admission | | |
| Mean BP | Mean, variance | No |
| Systolic BP | Mean | No |
| Diastolic BP | Mean | No |
| Heart rate | Mean, variance | No |
| Respiration rate | Mean, variance | No |
| Feature | Summary Measure | Categorized |
| SaO2 | Mean | No |
| Glucose | Mean | No |
| White blood cell | Mean | No |
| Creatinine | Mean | No |
| Hemoglobin | Mean | No |
| Admission source | NA | Observation, Acute care or floor, |
| | | floor, unspecified, ED, Recovery |
| | | room, Other hospital, ER, Other |
| | | ICU, Direct Admit, Chest Pain |
| | | center, PACU, ICU, SDU, OR |
| Admission diagnosis | NA | Yes |
| Glasgow Coma Scale | NA | Yes |
| (GCS) | | |
| pH | Mean | Yes |
| Lactate | Mean | Yes |
| Albumin | Mean | Yes |
| PaCO2 | Mean | Yes |

As shown in Table 1, the plurality of different defined ICU prediction features can include one or more different data inputs from one or more different data input categories. In some embodiments, the plurality of different defined ICU prediction features include multiple records for a data input taken over time. For example, the heart rate of the patient may be extracted over a period of time such that a mean and variability statistics can also be extracted. In other embodiments, the plurality of different defined ICU prediction features includes only a single record for a particular data input. For example, the extracted plurality of different defined ICU prediction features can include a total Glasgow Coma Scale (GCS) representative of the GCS assessed most recently during the first time period.

In embodiments, some of the defined ICU ventilation prediction features (e.g., admission source, admission diagnosis, etc.) may be summarized over the first period of time of ICU stay if available, while some less-commonly measured prediction features (e.g., lactate or pH, etc.) may be converted to categorical variables, as indicated above, including a “missing” category.

For example, certain health features and/or ICU ventilation prediction features may be highly informative predictors, but are measured infrequently. For such variables, a physiologically relevant binning scheme may be used, including a “missing” bin to indicate if the variable was missing. As discussed below, this binning scheme allows the ICU ventilation prediction model(s) 264 to learn from the “missingness” of the health and/or prediction feature without the necessity of imputation. In specific embodiments, the binning scheme can include at least the bins shown in Table 2:

TABLE 2
BINNING SCHEME EXAMPLE

| Health and/or Prediction Feature | Bins for Categorization |
|---|---|
| Blood albumin | <= 1.5 |
| | >1.5-3.5 |
| | >3.5-4.0 |
| | >4.0-4.5 |
| | >4.5-5.0 |
| | >5.0-100 |
| | Missing |
| Blood lactate | <= 0.5 |
| | >0.5-1 |
| | >1-2 |
| | >2-4 |
| | >4-50 |
| | Missing |
| Arterial blood gas, pH | <= 7.35 |
| | >7.35-7.40 |
| | >7.40-7.45 |
| | >7.45-8.00 |
| | Missing |
| Arterial blood gas, PaCO2 | <= 25 |
| | >25-35 |
| | >35-45 |
| | >45-60 |
| | >60-75 |
| | >75-200 |
| | Missing |
| Arterial blood gas, PaO2 | <= 100 |
| | >100-300 |
| | >300-650 |
| | Missing |
| GCS score | 3 |
| | >4-6 |
| | 7 |
| | 8 |
| | 9 |
| | 10 |
| | 11 |
| | 12 |
| | 13 |
| | 14 |
| | 15 |
| | Unable to score |
| | Missing |

However, the plurality of different defined ICU prediction features are not limited to only these features, and it is contemplated that other data input categories and other data inputs may be defined in future models. In particular embodiments, the prediction features may be defined to ensure a clinically accurate reflection of the patient.

In embodiments, the plurality of different defined ICU prediction features may be automatically extracted from the plurality of records obtained in step 120 using natural language processing and/or a machine learning algorithm. For example, the ICU ventilation prediction system 200 may include a prediction feature extractor 261 that implements a natural language processing technique and/or a machine learning algorithm in order to extract the plurality of different predefine ICU prediction features.

At a step 140, the method 100 includes analyzing the extracted plurality of different defined ICU prediction features using a trained ICU ventilation prediction model 264. In embodiments, the ICU ventilation prediction system 200 can apply the trained ICU ventilation prediction model(s) 264 to the extracted plurality of different defined ICU prediction features.

In embodiments, the extracted plurality of different predefine ICU prediction features may be analyzed by fitting the prediction features to the trained ICU ventilation prediction model(s) 264. In some embodiments, the trained ICU ventilation prediction models 264 include at least a first model configured to predict how long a patient would be ventilated using either invasive ventilation and/or non-invasive ventilation, as well as a second model configured to predict a duration of ventilation.

In some embodiments, one or more of ICU ventilation prediction model(s) 264 may be developed using a gradient-boosting regression machine learning framework that allows the ICU ventilation prediction model 264 to capture non-linear relationships between input features and the ventilation duration, as well as allowing the ICU ventilation prediction model 264 to learn interactions between features and while also providing great interpretability.

In other embodiments, one or more of ICU ventilation prediction model(s) 264 may be developed using a multiclass gradient boosting model configured to predict whether a patient belongs to one of multiple classes (e.g., four classes, etc.) of ventilation status at any time during their ICU stay. For example, these multiple classes may the following classes shown in Table 3:

TABLE 3
VENTILATION PROBABILITY CLASSES

| Class | Definition |
|---|---|
| 1 | None: patient receiving neither invasive nor |
| | non-invasive ventilation |
| 2 | Non-invasive only: patient receiving only non-invasive |
| | ventilation but not invasive ventilation |
| 3 | Invasive only: patient receiving only invasive |
| | ventilation but not non-invasive ventilation |
| 4 | Both: patient receiving both invasive and |
| | non-invasive ventilation |

In further embodiments, the trained ICU ventilation prediction model(s) 264 may enable analysis of the extracted plurality of different defined ICU prediction features even when one or more defined prediction features are missing from the extracted prediction features for the patient. For example, in some embodiments, one or more variables that are less commonly measured at ICU admission may be missing from the patient's medical records (and therefore not included in the patient's records received in step 120). In specific embodiments, the trained ICU ventilation prediction model(s) 264 may be used even though one or more of the data inputs listed in Table 1 above are missing.

At a step 150, the method 100 includes generating a likelihood of ventilation for the patient based on the analysis performed in step 140. In embodiments, the likelihood of ICU ventilation can include at least one of: (i) a prediction of invasive and/or non-invasive ventilation of the patient in the ICU; and/or (ii) a predicted duration of invasive and/or non-invasive ventilation of the patient in the ICU.

In specific embodiments, the prediction of invasive and/or non-invasive ventilation of the patient in the ICU comprises a prediction of one or more of: (i) ventilation versus no ventilation; (ii) invasive ventilation versus no invasive ventilation; and/or (iii) non-invasive ventilation versus no non-invasive ventilation. In some embodiments, these predicted probabilities may be generated by summing the predicted probabilities for the component classes.

In further specific embodiments, the predicted duration of invasive and/or non-invasive ventilation of the patient in the ICU can include generating a determining a duration period, including but not limited to, less than 12 hours, 12 hours to 1 day, 1 day to 3 days, 3 days to 7 days, 7 days to 10 days, and/or more than 10 days.

At a step 160, the method 100 includes presenting the generated likelihood of ICU ventilation for the patient. For example, in embodiments, the generated likelihood of ICU ventilation for the patient may be presented to a healthcare worker, administrator, and/or provider responsible for the patient. In some embodiments, the generated likelihood of ICU ventilation for the patient may be presented via a user interface, such as a display screen or computer monitor. In embodiments, the user interface used to present the generated likelihood of ICU ventilation for the patient may be a user interface 240 of the ICU ventilation prediction system 200. In still further embodiments, the patient is still admitted to the ICU while the likelihood of ICU ventilation is generated and/or presented (e.g., the method 100 is performed before the patient is discharged from the ICU).

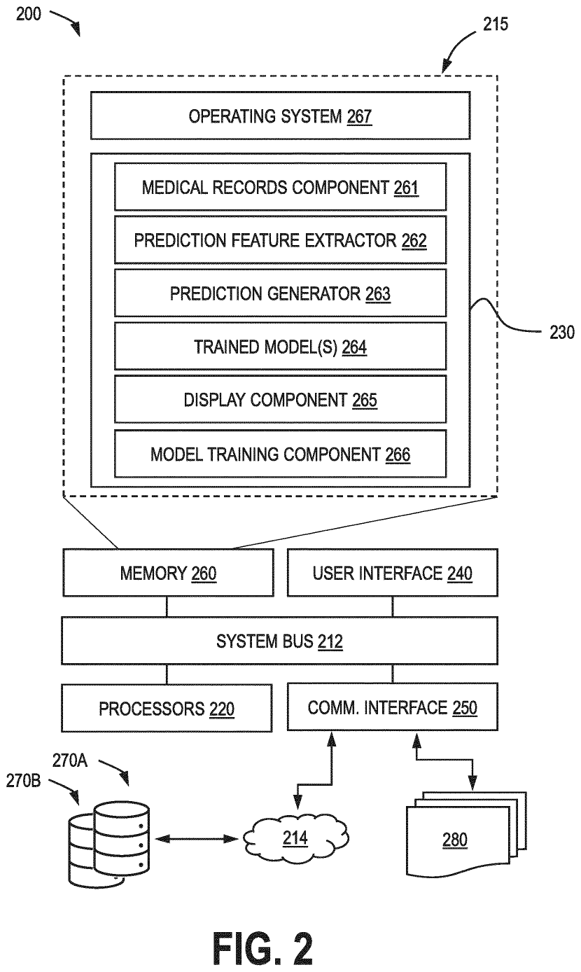

Turning to FIG. 2, an example ICU ventilation prediction system 200 is illustrated. The ICU ventilation prediction system 200 can be configured to predict a likelihood of ventilation for a patient, as described above. In some embodiments, the ICU ventilation prediction system 200 may be at least part of a larger patient data management system (PDMS) and/or a patient monitoring system.

In embodiments, the ICU ventilation prediction system 200 comprises one or more processors 220, machine-readable memory 260, a user interface 240, and/or a communications interface 250, all of which may be interconnected and/or communication through a system bus 212 containing conductive circuit pathways through which instructions (e.g., machine-readable signals) may travel to effectuate communication, tasks, storage, and the like.

As discussed in more detail below, the one or more processors 220 may be configured to perform one or more steps of the methods described herein, including but not limited to, the following: (i) obtain, from an electronic medical records database 270A, 270B, a plurality of records for one or more patients in an ICU covering at least a first time period; (ii) extract, from the obtained plurality of records, a plurality of different defined ICU prediction features for one or more patients; (iii) analyze the extracted plurality of different defined ICU prediction features using a trained ICU ventilation prediction model 264; and (iv) generate, from the analysis, a likelihood of ventilation for one or more patients.

In some examples, the one or more processors 220 may include a high-speed data processor adequate to execute the program components described herein and/or various specialized processing units as may be known in the art. In some examples, the one or more processors 220 may be a single processor, multiple processors, or multiple processor cores on a single die.

In some examples, the communications interface 250 can include a network interface configured to connect the ICU ventilation prediction system 200 to a communications network 214, an input/output (“I/O”) interface configured to connect and communicate with one or more peripheral devices, a memory interface configured to accept, communication, and/or connect to a number of machine-readable memory devices, and the like.

In certain embodiments, the communications interface 250 may operatively connect the ICU ventilation prediction system 200 to a communications network 214, which can include a direct interconnection, the Internet, a local area network (“LAN”), a metropolitan area network (“MAN”), a wide area network (“WAN”), a wired or Ethernet connection, a wireless connection, and similar types of communications networks, including combinations thereof. In some examples, ICU ventilation prediction system 200 may communicate with one or more remote/cloud-based servers (e.g., the electronic medical records database 270A), cloud-based services, and/or remote devices via the communications network 214.

The memory 260 can be variously embodied in one or more forms of machine-accessible and machine-readable memory. In some examples, the memory 260 includes a storage device that comprises one or more types of memory. For example, a storage device can include, but is not limited to, a non-transitory storage medium, a magnetic disk storage, an optical disk storage, an array of storage devices, a solid-state memory device, and the like, including combinations thereof.

Generally, the memory 260 is configured to store data/information and instructions 215 that, when executed by the one or more processors 220, causes the ICU ventilation prediction system 200 to perform one or more tasks. In particular examples, the memory 260 includes an ICU ventilation prediction package 230 that causes the ICU ventilation prediction system 200 to perform one or more steps of the methods described herein.

In embodiments, the ICU ventilation prediction package 230 comprises a collection of program components, database components, and/or data. Depending on the particular implementation, the ICU ventilation prediction package 230 may include software components, hardware components, and/or some combination of both hardware and software components.

The ICU ventilation prediction package 230 may include one or more software packages configured to predict a likelihood of ventilation for a patient. These software packages may be incorporated into, loaded from, loaded onto, or otherwise operatively available to and from the ICU ventilation prediction system 200.

In some examples, the ICU ventilation prediction package 230 and/or one or more individual software packages may be stored in a local storage device 260. In other examples, the ICU ventilation prediction package 230 and/or one or more individual software packages may be loaded onto and/or updated from a remote server via the communications interface 250.

In particular embodiments, the ICU ventilation prediction package 230 can include, but is not limited to, instructions 215 having a medical records component 261, prediction feature extractor 262, a prediction generator 263, one or more trained ICU ventilation prediction models 264, a display component 263, and/or a model training component 266. These components may be incorporated into, loaded from, loaded onto, or otherwise operatively available to and from the ICU ventilation prediction system 200.

In embodiments, the medical records component 260 can be a stored program component that is executed by at least one processor, such as the one or more processors 220 of the ICU ventilation prediction system 200. In particular, the medical records component 260 can be configured to interface with an electronic medical records database 270A in order to obtain a plurality of records for one or more patients, as described herein. That is, the medical records component 260 may be configured to request, receive, and/or otherwise obtain a plurality of medical records for one or more patients in an ICU.

In embodiments, one or more of the patients may be historical patients. In other embodiments, one or more of the patients may be current ICU patients. In still further embodiments, the medical records component 260 may obtain a plurality of records for a combination of historical and/or current ICU patients.

In embodiments, the prediction feature extractor 261 can be a stored program component that is executed by at least one processor, such as the one or more processors 220 of the ICU ventilation prediction system 200. In particular, the prediction extractor 261 can be configured to extract a plurality of different predefine ICU prediction features for a patient, as described herein. In particular, the prediction feature extractor 261 can be configured to extract predefine ICU prediction features from the plurality of records obtained from an electronic medical records database 270A using natural language processing and/or a machine learning algorithm.

In embodiments, the prediction generator 263 can be a stored program component that is executed by at least one processor, such as the one or more processors 220 of the ICU ventilation prediction system 200. In particular, the prediction generator 263 can be configured to analyze the extracted plurality of different predefine ICU prediction features and generate a likelihood of ventilation, as described herein.

In particular embodiments, the prediction generator 263 can be configured to use one or more trained ICU ventilation prediction model(s) 264 in order to analyze the extracted ICU prediction features. Based on the output of applying the one or more trained ICU ventilation prediction model(s) 264, the prediction generator 263 may generate a likelihood of ventilation for a particular patient.

In embodiments, the display component 265 can be a stored program component that is executed by at least one processor, such as the one or more processors 220 of the ICU ventilation prediction system 200. In particular, the display component 265 can be configured operate a user interface 240 in order to present the generated likelihood of ICU ventilation for the patient, as described herein. In some embodiments, the display component 265 can include a programmable processor, also referred to as a graphics progressing units (GPU), which is specialized for rendering images on a monitor or display screen of a user interface 240. In other words, the user interface 240 may be configured, via a display component 265, to provide or otherwise present a likelihood of ventilation generated for one or more patients.

The ICU ventilation prediction system 200 may also include an operating system component 267, which may be stored in the memory 260. The operating system component 267 may be an executable program facilitating the operation of the ICU ventilation prediction system 200. Typically, the operating system component 267 can facilitate access of the communications interface 250, and can communicate with other components of the ICU ventilation prediction system 200, including but not limited to, the user interface 240, the memory 260, and/or the electronic medical records database 270A.

According to certain embodiments, the ICU ventilation prediction system 200 includes at least an electronic medical records database 270A, 270B, a processor 220, a user interface 240, and a trained ICU ventilation prediction model 264. In embodiments, the ICU ventilation prediction model 264 may be trained by the training component 266 using a training dataset 280 comprising a plurality of records for each of a plurality of patients over a period of time covering each patient's stay in an ICU.

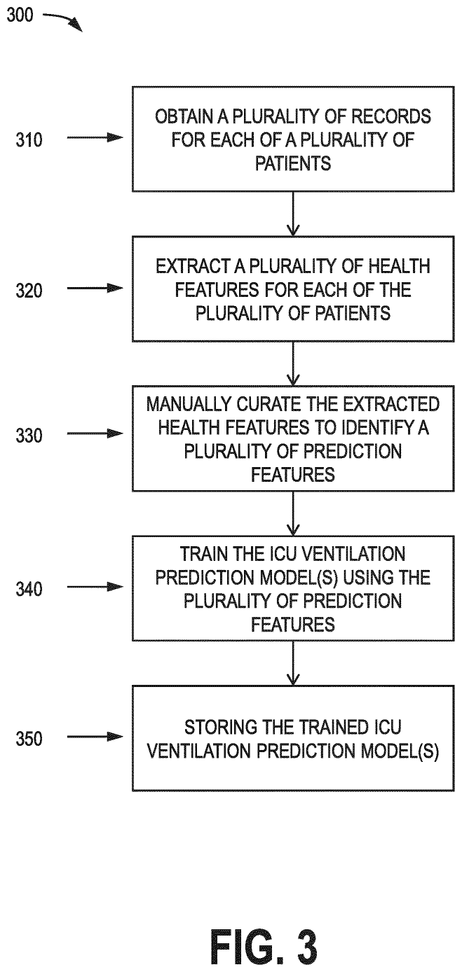

For example, with reference to FIG. 3, a flowchart of a method 300 for training an ICU ventilation prediction model is illustrated according to aspects of the present disclosure. In embodiments, the ICU ventilation prediction model may be trained by the ICU ventilation prediction system 200 and/or may be provided to the ICU ventilation prediction system 200 after having already been trained by another similar system.

At a step 310, the method 300 includes obtaining a training dataset 280 comprising a plurality of records for a plurality of patients. In embodiments, the plurality of records for the plurality of patients may be obtained from an electronic medical records database 270B. For example, the electronic medical records database 270B may include patient unit stays admitted to ICUs where physiologic, diagnosis, and treatment information (collectively, “medical information” or “medical data”) are captured, charted, or otherwise recorded. In embodiments, this may be the same electronic medical records database 270A, or may be a different electronic medical records database 270B. In embodiments, the use of the electronic medical records database 270 may be certified as necessary under regulatory and privacy standards.

In embodiments, the plurality of records obtained in step 310 can include medical records that cover at least a first period of time for each of the plurality of patients. For example, the plurality of records obtained in step 310 can include medical records that cover the first 24 hours of each patients' stay in an ICU (i.e., the medical data available through the first day of ICU admission). Longer and shorter time periods are possible. Alternatively, if medical records for one or more of the patients within the first day of ICU admission are not available, the medical records may include, for example and without limitation, medical records covering up to six hours before ICU admission, although this time period may be longer or shorter than six hours.

As such, in various examples, the first period of time covered by each of the plurality of medical records can include the first 24 hours of a patient's ICU stay, only the first 24 hours of a patient's ICU stay, less than 24 hours of a patient's ICU stay, an amount of time (e.g., 1 hour, 3 hours, 6 hours, 12 hours, etc.) preceding a patient's admission to the ICU, and/or some combination thereof.

In particular embodiments, for at least some of the plurality of records obtained, the corresponding patients in the ICU did not receive any ventilation, for at least some of the plurality records obtained, the corresponding patients in the ICU received non-invasive ventilation for a known non-invasive ventilation period, for at least some of the plurality of records obtained, the corresponding patients in the ICU received invasive ventilation for a known invasive ventilation period, and for at least some of the plurality of records obtained, the corresponding patients in the ICU received both invasive and non-invasive ventilation each for a known ventilation period.

At a step 320, the method 300 includes extracting a plurality of health features for each of the plurality of patients from the training dataset 280 obtained in step 310. In embodiments, these health features may be clinical features representing a patient's ICU stay.

For example, continuous features commonly measured (e.g., vital signs, chemistry labs, basic characteristics, etc.) may be included, as well as other continuous features less commonly measured (e.g., lactate, pH, etc.). Health features with many nominal values may be collapsed with cut points defined by clinical knowledge and data distribution to ensure clinically meaningful groups with large enough sample sizes to support stable coefficient estimation.

At a step 330, the method 300 includes curating the extracted plurality of health features in order to identify and define a set of ICU prediction features. That is, the extracted plurality of health features may be curated to identify and define the plurality of different defined ICU prediction features (such as the plurality of defined ICU prediction features using steps 130, 140 of a method 100). According to an embodiment, manually curating the extracted plurality of health features comprises an analysis of the extracted plurality of health features to identify which are likely, more likely, less likely, or unable to predict ventilation status of a patient in the ICU. The manual curation can be done by a clinician, machine learning specialist, or any other person capable of reviewing an extracted health feature and evaluating the impact of the feature on the prediction of ventilation status of a patient in the ICU.

In embodiments, the step 330 can include binning one or more of the different defined ICU prediction features using a binning scheme, such as the binning scheme outlined in Table 2 above. In embodiments, the step 330 can include manually curating and/or binning one or more of the plurality of different health features extracted in step 320.

At a step 340, the method 300 includes training the ICU ventilation prediction model using the plurality of different defined ICU prediction features curated in step 330. In embodiments, the ICU ventilation prediction model may be trained using a plurality of different defined ICU prediction features corresponding to at least some of the plurality of patients for which medical records were obtained in step 310 (i.e., the training dataset 280).

At a step 350, the method 300 includes storing the trained ICU ventilation prediction model 264. In embodiments, the trained ICU ventilation prediction model 264 may be stored in the memory 260 of an ICU ventilation prediction system 200. In other embodiments, the trained ICU ventilation prediction model 264 may be stored remotely from an ICU ventilation prediction system 200, such as in a remote database accessible by an ICU ventilation prediction system 200 (e.g., via communications interface 250 and network 214).

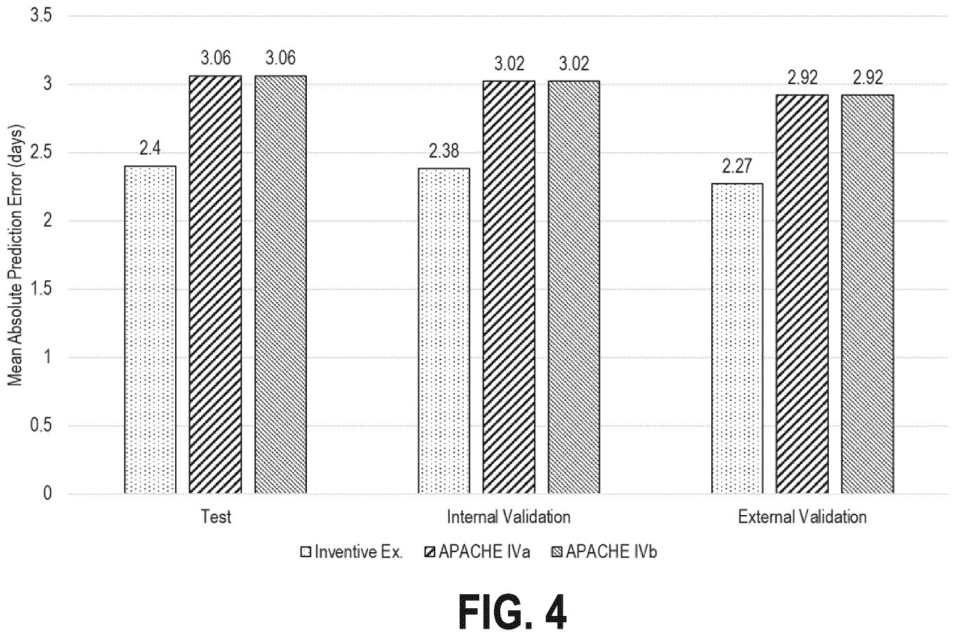

As described herein, the methods and systems of predicting a likelihood of ventilation for a patient achieve improved performance over existing approaches. For example, with reference to FIG. 4, the performance of an ICU ventilation prediction model 264 configured to predict a duration of ventilation as described herein is illustrated. As shown, the ICU ventilation prediction model 264 exhibits a consistently lower mean absolute prediction error when compared with two existing prediction models, APACHE IVa and APACHE IVb.

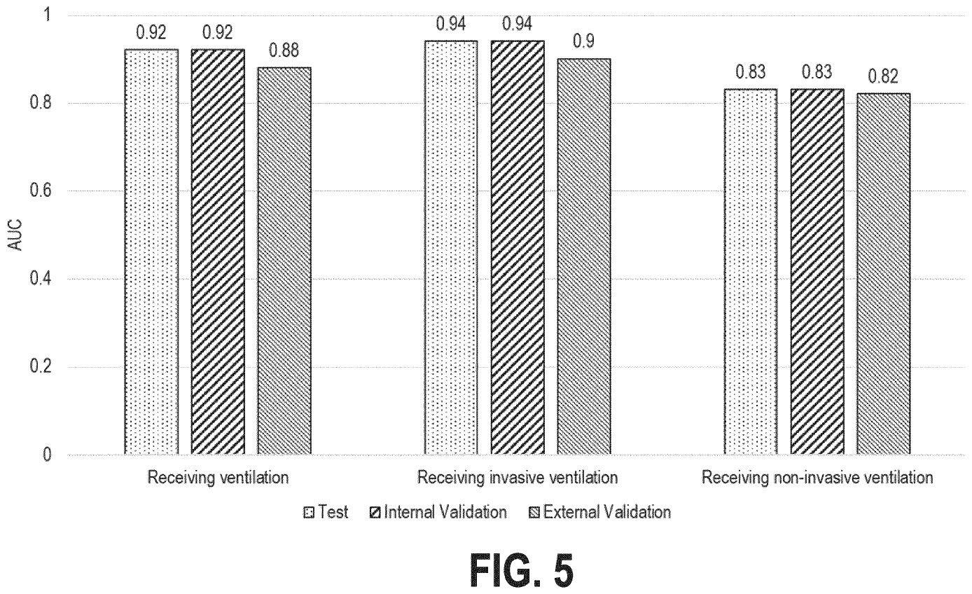

Further, with reference to FIG. 5, the performance of an ICU ventilation prediction model 264 configured to predict a probability of ventilation as described herein is illustrated. In particular, the performance of the ICU ventilation prediction model 264 for identifying patient receiving any ventilation (left), patients receiving any invasive ventilation (middle), and patients receiving any non-invasive ventilation (right), is shown according to the area under the receiver operating characteristic (AUC) measurement. As seen in FIG. 5, the ICU ventilation prediction model 264 exhibits a high degree of accuracy between the test dataset, internal validation dataset, and an external validation dataset.

According to an embodiment, the ICU ventilation prediction system is configured to process many thousands or millions of datapoints to extract the plurality of different defined ICU ventilation prediction features, to generate the likelihood of ICU ventilation for the patient, and to display the likelihood of ICU ventilation for the patient to a user via the user interface. Further, preferably data for 100s or 1000s of patients are used to train the ICU ventilation prediction model 264. Accordingly, the ICU ventilation prediction system is configured to process millions of datapoints to extract the plurality of different defined ICU ventilation prediction features for these 100s or 1000s of patients and use that data to train the ICU ventilation prediction model 264. This requires millions or billions of calculations, which a human mind could not perform in a lifetime. Further, since training the ICU ventilation prediction model 264 utilizes a unique data set, the stored trained ICU ventilation prediction model is a novel model.

By providing improved prediction of the likelihood of ICU ventilation for a patient, this novel ICU ventilation prediction system has an enormous positive effect on patient care compared to prior art systems. Improved understanding of the likelihood of ICU ventilation for a patient can improve patient care and health, thereby saving lives.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for predicting a likelihood of ventilation of a patient in an intensive care unit (ICU), the method comprising:
obtaining, from an electronic medical records database, a plurality of records for the patient in the ICU covering at least a first time period;
extracting, from the obtained plurality of records, a plurality of different defined ICU ventilation prediction features for the patient;
analyzing the extracted plurality of different defined ICU ventilation prediction features using a trained ICU ventilation prediction model; and
predicting, by the trained ICU ventilation prediction model, (i) a likelihood of a need for a ventilation of the patient in the ICU; and (ii) a duration of the need for the ventilation of the patient in the ICU.

2. The method of claim 1, further comprising:
predicting, by the trained ICU ventilation prediction model, one or more of: (i) no ventilation; (ii) invasive ventilation only; (iii) non-invasive ventilation only; and
(iv) both invasive and non-invasive ventilation.

3. The method of claim 1, further comprising:
predicting, by the trained ICU ventilation prediction model, one or more of: (i) ventilation versus no ventilation; (ii) invasive ventilation versus no invasive ventilation; and
(iii) non-invasive ventilation versus no non-invasive ventilation.

4. The method of claim 1, wherein the extracted plurality of different defined ICU ventilation prediction features for the patient comprises one or more of BMI, age, gender, pre-ICU admission lead time, ventilation status, admitted with elective surgery status, mean blood pressure, systolic blood pressure, diastolic blood pressure, heart rate, respiratory rate, oxygen saturation, blood glucose, white blood cell count, blood sodium, blood potassium, blood creatinine, blood hemoglobin, blood albumin, blood lactate, arterial blood gas, admission diagnosis, and Total Glasgow Coma Scale.

5. The method of claim 1, wherein the predicting is performed by an ICU ventilation prediction system that is, a component of, a patient data management systems (PDMS) or a patient monitoring system.

6. The method of claim 1, wherein obtaining, from an electronic medical records database, a plurality of records for each of a plurality of patients in an ICU comprises obtaining a plurality of records for a plurality of historical ICU patients.

7. The method of claim 1, wherein the ICU ventilation prediction model is a gradient-boosted decision tree model.

8. The method of claim 1, wherein the trained ICU ventilation prediction model is configured to analyze the extracted plurality of different defined ICU ventilation prediction features and predict a likelihood of the need for the ventilation of the patient in the ICU when at least one of the plurality of different ICU ventilation prediction features are missing from the obtained plurality of records.

9. The method of claim 16, further comprising binning at least one of the extracted plurality of different health features into bins prior to training the ICU ventilation prediction model, and wherein the bins comprise a bin comprising missing data.

10. The method of claim 91, wherein the binned extracted plurality of different health features comprise one or more of blood albumin, blood lactate, arterial blood gas pH, arterial blood gas PaCO₂, arterial blood gas PaO₂, PF ratio, and Glasgow Coma Scale (GCS) score.

11. An intensive care unit (ICU) ventilation prediction system configured to predict a likelihood of ventilation of a patient in an ICU, the system comprising:
an electronic medical records database comprising a plurality of records for a plurality of patients; and
a processor configured to:
obtain, from the electronic medical records database, a plurality of records for the patient in the ICU covering at least a first time period;
extract, from the obtained plurality of records, a plurality of different defined ICU ventilation prediction features for the patient;
analyze the extracted plurality of different defined ICU ventilation prediction features using a trained ICU ventilation prediction model; and
predict, by the trained ICU ventilation prediction model, (i) a likelihood of a need for a ventilation of the patient in the ICU; and (ii) a duration of the need for the ventilation of the patient in the ICU.

12. The system of claim 11, wherein the the processor is further configured to predict:
one or more of: (i) no ventilation; (ii) invasive ventilation only; (iii) non-invasive ventilation only; and (iv) both invasive and non-invasive ventilation; or
one or more of: (i) ventilation versus no ventilation; (ii) invasive ventilation versus no invasive ventilation; and (iii) non-invasive ventilation versus no non-invasive ventilation.

13. The system of claim 11, wherein the extracted plurality of different defined ICU ventilation prediction features for the patient comprises one or more of BMI, age, gender, pre-ICU admission lead time, ventilation status, admitted with elective surgery status, mean blood pressure, systolic blood pressure, diastolic blood pressure, heart rate, respiratory rate, oxygen saturation, blood glucose, white blood cell count, blood sodium, blood potassium, blood creatinine, blood hemoglobin, blood albumin, blood lactate, arterial blood gas, admission diagnosis, or Total Glasgow Coma Scale.

14. The system of claim 11, wherein the ICU ventilation prediction system is a component of, a patient data management systems (PDMS) or a patient monitoring system.

15. The system of claim 17, wherein the second processor is further configured to bin at least one of the extracted plurality of different health features into bins prior to training the ICU ventilation prediction model, and wherein the bins comprise a bin comprising missing data.

16. The method of claim 1, wherein the ICU ventilation prediction model is trained by:
(i) obtaining, from an electronic medical records database, a plurality of historical records for each of a plurality of historical patients in an ICU, wherein at least one of the plurality of historical patients in the ICU did not receive any ventilation, and wherein at least one of the plurality of historical patients in the ICU received non-invasive ventilation for a known non-invasive ventilation period, and wherein at least one of the plurality of historical patients in the ICU received invasive ventilation for a known invasive ventilation period, and wherein at least one of the plurality of historical patients in the ICU received both invasive and non-invasive ventilation each for a known ventilation period;
(ii) extracting, from the obtained plurality of historical records, a plurality of different health features for each of the plurality of historical patients;
(iii) curating the extracted plurality of different health features to identify a plurality of different historical ICU ventilation prediction features;
(iv) training the ICU ventilation prediction model using the plurality of different historical ICU ventilation prediction features; and
(v) storing the trained ICU ventilation prediction model.

17. The method of claim 1, further comprising:
presenting, via a user interface, at least one of (i) the predicted likelihood of the need for the ventilation of the patient in the ICU; and (ii) the predicted duration of the need for the ventilation of the patient in the ICU.

18. The system of claim 11, wherein a second processor is configured to train the ICU ventilation prediction model, the second processor is configured to:
obtain, from an electronic medical records database, a plurality of historical records for each of a plurality of historical patients in an ICU, wherein at least one of the plurality of historical patients in the ICU did not receive any ventilation, and wherein at least one of the plurality of historical patients in the ICU received non-invasive ventilation for a known non-invasive ventilation period, and wherein at least one of the plurality of historical patients in the ICU received invasive ventilation for a known invasive ventilation period, and wherein at least one of the plurality of historical patients in the ICU received both invasive and non-invasive ventilation each for a known ventilation period;
extract, from the obtained plurality of historical records, a plurality of different health features for each of the plurality of historical patients;
curate the extracted plurality of different health features to identify a plurality of different historical ICU ventilation prediction features;
train the ICU ventilation prediction model using the plurality of different historical ICU ventilation prediction features; and
store the trained ICU ventilation prediction model.

19. The system of claim 11, further comprising:
a user interface configured to provide at least one of (i) the predicted likelihood of the need for the ventilation of the patient in the ICU; and (ii) the predicted duration of the need for the ventilation of the patient in the ICU.

20. A non-transitory computer-readable storage medium having stored a computer program comprising instructions, which, when executed by at least one processor, cause the at least one processor to:
obtain, from an electronic medical records database, a plurality of records for a patient in an ICU covering at least a first time period;
extract, from the obtained plurality of records, a plurality of different defined ICU ventilation prediction features for the patient;
analyze the extracted plurality of different defined ICU ventilation prediction features using a trained ICU ventilation prediction model; and
predict, by the trained ICU ventilation prediction model, (i) a likelihood of a need for a ventilation of the patient in the ICU; and (ii) a duration of the need for the ventilation of the patient in the ICU.
